(19) United States
(12) Patent Application Publication
(10) Pub. No.: US 2018/0353664 A1
(43) Pub. Date: Dec. 13, 2018

(54) SUCTION COLLAR FOR ELECTROSURGICAL DEVICES

(71) Applicant: Innoblative Designs, Inc., Chicago, IL (US)

(72) Inventors: Robert F. Rioux, Ashland, MA (US); Ryan M. Bean, Westminster, MA (US); Tyler Wanke, Chicago, IL (US); V. Suzanne Klimberg, Little Rock, AR (US)

(21) Appl. No.: 16/001,494

(22) Filed: Jun. 6, 2018

Related U.S. Application Data
(60) Provisional application No. 62/516,374, filed on Jun. 7, 2017.

(51) Int. Cl. A61M 1/00; A61B 18/14

(52) U.S. Cl. CPC A61M 1/0088; A61B 2017/00862; A61B 18/148

(57) ABSTRACT

The invention is an accessory configured to be fitted to an electrical surgical device and provide suction at a surgical site in which the electrical surgical device is being used. In particular, the accessory is in the form of a suction collar configured to be releasably coupled to the electrical surgical device, generally adjacent to a working end thereof, and provide suction to minimize the accumulation of excess steam, fluid, and/or debris from the surgical site during the treatment procedure. The suction collar is further configured to apply a sufficient suction force to a patient's skin surrounding the surgical site to aid in stabilizing the electrosurgical device in place and maintain the working end to the electrosurgical device within the surgical site during the procedure.

### CROSS-REFERENCE TO RELATED APPLICATIONS

This application claims the benefit of, and priority to, U.S. Provisional Application No. 62/516,374, filed Jun. 7, 2017, the content of which is hereby incorporated by reference herein in its entirety.

### FIELD

The present disclosure relates generally to medical devices, and, more particularly, to a suction accessory configured to be fitted to an electrosurgical device and provide suction to minimize the accumulation of excess steam, smoke, fluid, and/or debris from a surgical site during a treatment procedure and further aid in stabilizing the device during the procedure.

### BACKGROUND

Cancer is a group of diseases involving abnormal cell growth with the potential to invade or spread to other parts of the body. Cancer generally manifests into abnormal growths of tissue in the form of a tumor that may be localized to a particular area of a patient's body (e.g., associated with a specific body part or organ) or may be spread throughout. Tumors, both benign and malignant, are commonly treated and removed via surgical intervention, as surgery often offers the greatest chance for complete removal and cure, especially if the cancer has not spread to other parts of the body. Electrosurgical methods, for example, can be used to destroy these abnormal tissue growths. However, in some instances, surgery alone is insufficient to adequately remove all cancerous tissue from a local environment.

For example, treatment of early stage breast cancer typically involves a combination of surgery and adjuvant irradiation. Unlike a mastectomy, a lumpectomy removes only the tumor and a small rim (area) of the normal tissue around it. Radiation therapy is given after lumpectomy in an attempt to eradicate cancer cells that may remain in the local environment around the removed tumor, so as to lower the chances of the cancer returning. However, radiation therapy as a post-operative treatment suffers various shortcomings. For example, radiation techniques can be costly and time consuming, and typically involve multiple treatments over weeks and sometimes months. Furthermore, radiation often results in unintended damage to the tissue outside the target zone. Thus, rather than affecting the likely residual tissue, typically near the original tumor location, radiation techniques often adversely affect healthy tissue, such as short and long-term complications affecting the skin, lungs, and heart.

Accordingly, such risks, when combined with the burden of weeks of daily radiation, may drive some patients to choose mastectomy instead of lumpectomy. Furthermore, some women (e.g., up to thirty percent (30%)) who undergo lumpectomy stop therapy before completing the full treatment due to the drawbacks of radiation treatment. This may be especially true in rural areas, or other areas in which patients may have limited access to radiation facilities.

### SUMMARY

Tumors, both benign and malignant, are commonly treated and destroyed via surgical intervention, as surgery often offers the greatest chance for complete removal and cure, especially if the cancer has not metastasized. However, after the tumor is destroyed, a hollow cavity may remain, wherein tissue surrounding this cavity and surrounding the original tumor site can still leave abnormal or potentially cancerous cells that the surgeon fails, or is unable, to excise. This surrounding tissue is commonly referred to as “margin tissue” or “marginal tissue”, and is the location within a patient where a reoccurrence of the tumor may most likely occur.

Some alternative treatments to using radiation therapy include the use of electrosurgical devices, including ablation devices, to be inserted within cavitary excisional beds and deliver radiofrequency (RF) energy to marginal tissue surrounding the cavity following the procedure. For example, one type of ablation applicator includes a long rigid needle-based electrode applicator for delivery of RF energy to marginal tissue upon manual manipulation by a surgeon or operator. Another type of ablation applicator includes an umbrella-type array of electrodes jointly connected to one another and deployable in an umbrella-like fashion to deliver RF energy. Yet still, another type of ablation applicator includes a probe having either a round rigid applicator head or a deployable applicator head, generally in the form an expandable balloon, configured to be placed within the tissue cavity (e.g., formed from the tumor removal surgery) and ablate marginal tissue via RF from an electrode array disposed about the probe surface. The treatment procedure may further include the use of a conductive fluid, such as saline, provided through the applicator head and to the surrounding marginal tissue so as to carry electrical current from electrode array to the tissue thereby creating a virtual electrode.

During certain medical treatments, it may be necessary to evacuate accumulating fluids or debris from the region of treatment. For example, during a procedure involving ablation devices for the destruction of thin rim of marginal tissue around a cavity, from which a tumor has been surgically removed, excess fluid in the form of blood, interstitial fluid, as well as saline (i.e., as a result of the virtual electrode arrangement), as well as excess tissue debris, may result. Furthermore, the ablation procedure can generate steam, vapors, and smoke from heated or burnt tissue. When this occurs, visualization of the surgical site can become obscured, leading to potential dangerous conditions for the patient.

The present invention is an accessory configured to be fitted to an electrosurgical device, such as an ablation applicator, and provide suction at the surgical site in which the electrosurgical device is operating. In particular, the accessory is in the form of a collar configured to be releasably coupled to the electrosurgical device, generally positioned around the handle or neck portion of the device adjacent to a working end thereof, and provide suction for removal of excess accumulation of steam, smoke, fluid, and/or debris from the surgical site during the treatment procedure. The collar is further configured to apply a sufficient suction force to a patient's skin surrounding the surgical site to aid in stabilizing the electrosurgical device in place and maintain the working end to the electrosurgical device within the surgical site during the procedure.

In particular, the suction collar of the present disclosure includes a ring-shaped body configured to be positioned around a handle and/or neck portion of an electrosurgical device and a proximal end extending from the body and defining a port outlet configured to be coupled to a vacuum source (i.e., operating room wall vacuum or standalone vacuum pump) via a connection line (i.e., medical grade tubing or the like). The ring-shaped body includes at least a first body member extending from the proximal end and forming a first semi-circular half of the body and a second body member extending from the proximal end and a forming a second semi-circular half of the body complementary to the first semi-circular half. The ring-shaped body further includes a central aperture or orifice enclosed by the first and second body members and shaped and/or sized to receive a neck portion of the electrosurgical device to thereby allow the suction collar to be coupled to the device. While the first and second body members generally extend from and share a common point at the proximal end, each of the first and second body members are independent of one another at respective distal ends (i.e., not fixed to one another and can be separated). The suction collar is formed from a flexible or deformable material, such as an elastomer, which allows for the first and second body members to be manipulated and separated from one another when a user (i.e., surgeon or other medical professional) is either attaching or removing the suction collar to or from an electrosurgical device. In particular, a user need only separate the first and second body members from one another to thereby expose the central aperture or orifice, at which point the body can be mounted to a handle or neck portion of the electrosurgical device. Once the body is positioned onto the electrosurgical device, the user can simply allow the first and second body members to return to their default shapes (i.e. semi-circular halves), at which point the first and second body member will fully enclose the aperture and the electrosurgical device within, thereby securing the suction collar to the electrosurgical device.

Each of the first and second body members are generally hollow and each includes a pathway extending from the distal end thereof to the port outlet of the proximal end of the suction collar. In particular, the port outlet is in fluid communication with the pathways of the first and second body members. Furthermore, each of the first and second body members includes at least a first set of perforations, apertures, or holes defined on a bottom surface thereof. Generally, the bottom surface of the collar body is configured to be oriented in a direction facing the surgical site, and, in some instances, further engage portions of the surgical site. Accordingly, upon activation of the vacuum source, suction is created at each of the apertures, and, in turn, fluid, steam, smoke, and/or debris from the surgical site may then flow into the suction collar, by way of one or more of the apertures, flow through the pathway of at least one of the first and second body members of the collar body, and eventually flow out of the suction collar via the port outlet. In some embodiments, each of the first and second body members may further include a second set of perforations generally defined on an inner circumference of each of the first and second body members, generally positioned closer to the central aperture or orifice than the first set of aperture. Similar to the first set of apertures, suction can be created at each of the second set of perforations upon activation of the vacuum source.

In addition to aspirating fluid, steam, smoke, and/or debris, the suction collar is further configured to apply a sufficient suction force to a patient's skin surrounding the surgical site to aid in stabilizing the electrosurgical device in place and maintain the working end to the electrosurgical device within the surgical site during the procedure. For example, in the event that a surgeon is ablating marginal tissue within a cavity with an ablation applicator, the surgeon can utilize the suction collar to not only minimize steam, smoke, excess saline, as well as debris, such a liquefied fat, from the ablation site (i.e., tissue cavity), but to further stabilize and maintain the applicator head within the cavity as a result of suction applied to the patient's skin surrounding the incision leading to the cavity. For example, the bottom surface of the body of the suction collar can directly engage the patient's skin, and, upon activation of the vacuum source, suction at the first set of perforations is sufficient to hold the suction collar in place, and, in turn, stabilizes the ablation applicator.

The suction collar of the present disclosure provides numerous advantages. The suction collar facilitates the ease of operation and makes it possible for a single user to perform certain tasks or treatments without the help of an assistant. In particular, some known medical procedures may often require an assistant (i.e., fellow surgeon, physician assistant, nurse, etc.) to handle and manipulate additional instruments during a procedure, while the surgeon handles electrosurgical device which may require the surgeon to use both hands for the treatment. The suction collar is an accessory configured to be releasably mounted on the electrosurgical device, specifically mounted adjacent to the working end (i.e., applicator head) of the device, thereby allowing for aspiration of fluid, steam, smoke, and/or debris in an efficient manner, as the suction collar movement directly corresponds to movement and manipulation of the electrosurgical device. The suction collar further provides for stabilization of the electrosurgical device, thereby providing improved accuracy and safety during an ablation procedure. Furthermore, because the suction collar is not integrally formed as part of the electrosurgical device itself, a surgeon can simply remove the suction collar when aspiration and/or stabilization of the electrosurgical device is not desired. Accordingly, a suction collar consistent with the present disclosure may be well suited for use with electrosurgical devices, particularly for use with ablation devices that may rely on the use of conductive fluids (e.g., saline) for the ablation procedure and that will further generate steam, vapors, and smoke from heated or burnt tissue, specifically marginal tissue that has been ablated within a cavity an effort to manage residual disease within.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIGS. 1A and 1B are schematic illustrations of an ablation system consistent with the present disclosure;

FIG. 2 is a perspective view of an embodiment of a suction collar separated from and configured to be mounted to an exemplary ablation device the ablation system of FIG. 1A;

FIG. 3 is a perspective view of the suction collar mounted on the ablation device of FIG. 2, further illustrating the suction collar coupled to a vacuum source via a connection line;

FIG. 4 is a top perspective view of the suction collar of FIG. 2;

FIG. 5 is a bottom perspective view of the suction collar of FIG. 2;

FIGS. 6 and 7 are top and bottom plan views of the suction collar of FIG. 2;

FIG. 8 is a front view of the suction collar of FIG. 2;

FIG. 9 is a perspective sectional view of the suction collar of FIGS. 6 and 7 taken along lines A-A illustrating the internal pathway from the perforations to the port outlet;

FIG. 10 is a perspective section view of the suction collar of FIG. 8 taken along lines B-B illustrating the internal pathway from the perforations to the port outlet;

FIG. 11 is a side view of the suction collar mounted to the ablation device and illustrating suction via the perforations of the suction collar;

FIG. 12 illustrates placement of the applicator head within a tissue cavity and positioning of the suction collar adjacent to the surgical site for subsequent aspiration of fluids, steam, smoke, and/or debris from the surgical site and/or stabilizing of the ablation device based on suction of the skin surrounding the incision of the tissue cavity;

FIG. 13 illustrates placement of the applicator head within a tissue cavity and positioning of the suction collar adjacent to the surgical site and further illustrates suturing of the suction collar to the surrounding skin;

FIGS. 14A and 14B are bottom perspective views of a suction collar having a substantially planar bottom surface and a substantially concave bottom surface, respectively; and

FIGS. 15A and 15B are top perspective views of a suction collar illustrating one embodiment of the cooperating engagement between distal ends of the first and second body members of the suction collar and the separation of each from one another.

FIG. 16 is a top perspective view of a suction collar having at least a third set of perforations.

FIGS. 17 and 18 are top perspective and side views, respectively, of a suction collar having a body member comprised of a two-piece molded construction.

FIG. 19 is an enlarged top perspective view of the suction collar of FIGS. 17 and 18 illustrating a non-radial, substantially linear separation between distal most ends of first and second body members.

FIG. 20 is a bottom exploded perspective view of the suction collar of FIGS. 17 and 18 illustrating the two-piece molded construction, specifically an upper member and a lower member separated from one another.

FIG. 21 is a perspective view, partly in section, illustrating the coupling of the upper and lower members to one another to form the body member of the suction collar.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure is generally directed to a suction accessory configured to be fitted to an electrosurgical device and provide suction to minimize the accumulation of excess steam, fluid, and/or debris from a surgical site during a treatment procedure and further aid in stabilizing the device during the procedure.

In particular, the present disclosure is directed to an accessory configured to be fitted to an electrosurgical device, such as an ablation applicator, and provide suction at the surgical site in which the electrosurgical device is operating. In the embodiments described herein, the suction accessory is compatible for use with an ablation device to be used for an ablation procedure to destroy a thin rim of marginal tissue within a tissue cavity in an effort to manage residual disease in the local environment that has been treated. For example, the ablation device may be used for treating hollow body cavities, such as irregularly-shaped cavities in breast tissue created by a lumpectomy procedure. It should be noted, however, that the ablation device is not limited to such post-surgical treatments and, as used herein, the phrase “body cavity” may include non-surgically created cavities, such as natural body cavities and passages, such as the ureter (e.g. for prostate treatment), the uterus (e.g. for uterine ablation or fibroid treatment), fallopian tubes (e.g. for sterilization), and the like. Additionally, or alternatively, tissue ablation devices of the present disclosure may be used for the ablation of marginal tissue in various parts of the body and organs (e.g., skin, lungs, liver, pancreas, etc.) and is not limited to treatment of breast cancer.

The accessory is generally in the form of a collar configured to be releasably coupled to the ablation device, generally positioned around the handle or neck portion of the device adjacent to a working end thereof, and provide suction for removal of excess accumulation of steam, fluid, and/or debris from the surgical site during the treatment procedure. The collar is further configured to apply a sufficient suction force to a patient's skin surrounding the surgical site to aid in stabilizing the ablation device in place and maintain the working end (i.e., applicator head) of the device within the tissue cavity during the procedure.

The suction collar of the present disclosure provides numerous advantages. The suction collar facilitates the ease of operation and makes it possible for a single user to perform certain tasks or treatments without the help of an assistant. In particular, some known medical procedures may often require a separate assistant to handle and manipulate additional instruments during a procedure, while the surgeon handles electrosurgical device which may require the surgeon to use both hands for the treatment. The suction collar is an accessory configured to be releasably mounted on the electrosurgical device, specifically mounted adjacent to the working end (i.e., applicator head) of the device, thereby allowing for aspiration of fluid, steam, smoke, and/or debris in an efficient manner, as the suction collar movement directly corresponds to movement and manipulation of the electrosurgical device. The suction collar further provides for stabilization of the electrosurgical device, thereby providing improved accuracy and safety during an ablation procedure. Furthermore, because the suction collar is not integrally formed as part of the electrosurgical device itself, a surgeon can simply remove the suction collar when aspiration and/or stabilization of the electrosurgical device is not desired. Accordingly, a suction collar consistent with the present disclosure may be well suited for use with electrosurgical devices, particularly for use with ablation devices that may rely on the use of conductive fluids (e.g., saline) for the ablation procedure and that will further generate steam, vapors, and smoke from heated or burnt tissue, specifically marginal tissue that has been ablated within a cavity an effort to manage residual disease within.

The suction collar of the present disclosure is configured to be compatible with a wide variety of electrosurgical devices and is not limited to use with an ablation applicator/device described herein. However, for sake of clarity and ease of description, the following describes an ablation system and ablation device with which the suction collar may be used.

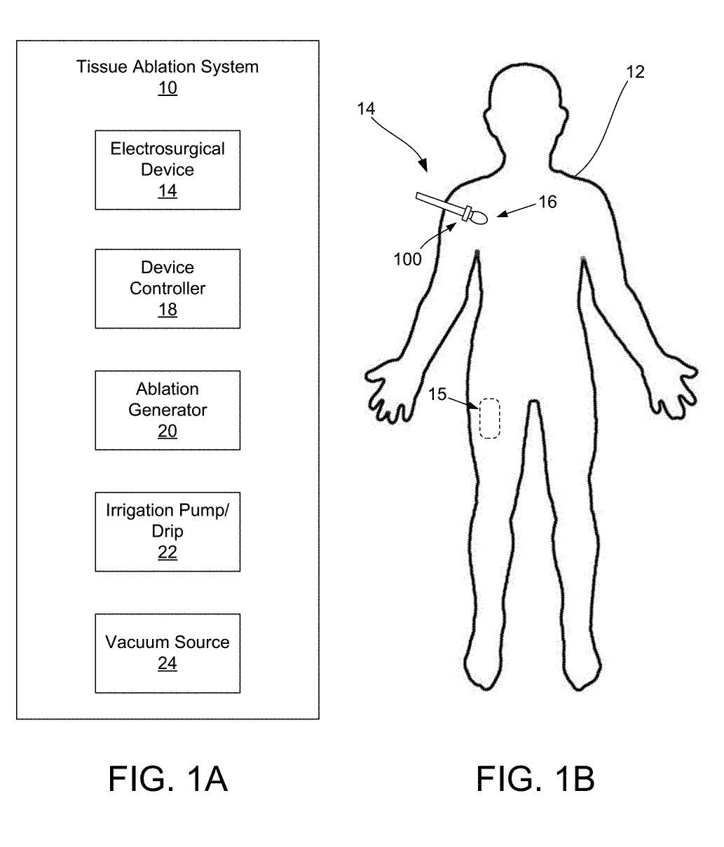

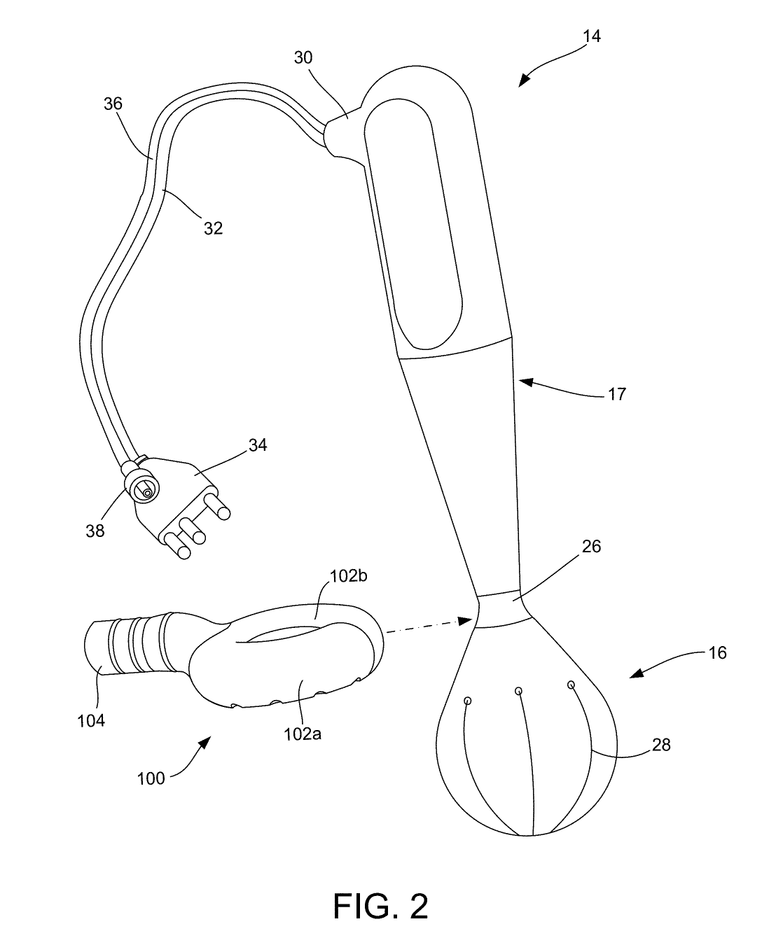

FIGS. 1A and 1B are schematic illustrations of an ablation system 10 for providing ablation of marginal tissue during a tumor removal procedure in a patient 12. The ablation system 10 generally includes an ablation device 14, which includes a probe having a distal tip or portion 16 and an elongated catheter shaft to which the distal tip 16 is connected. The catheter shaft may generally include a nonconductive elongated member including a fluid delivery lumen. The ablation device 14 may further be coupled to a device controller 18 and an ablation generator 20 over an electrical connection (electrical line 32 shown in FIG. 2), and an irrigation pump or drip 22 over a fluid connection (fluid line 36 shown in FIG. 2).

As will be described in greater detail herein, the device controller 18 may be used to control the emission of energy from one or more conductive elements of the device 14 to result in ablation, as well as controlling the delivery of fluid to the distal tip 16. In some cases, the device controller 18 may be housed within the ablation device 14. The ablation generator 20 may also connected to a return electrode 15 that is attached to the skin of the patient 12.

During an ablation treatment, the ablation generator 20 may generally provide RF energy (e.g., electrical energy in the radiofrequency (RF) range (e.g., 350-800 kHz)) to an electrode array of the ablation device 14, as controlled by the device controller 18. At the same time, saline may also be released from the distal tip 16. The RF energy travels through the blood and tissue of the patient 12 to the return electrode 15 and, in the process, ablates the region(s) of tissues adjacent to portions of the electrode array that have been activated.

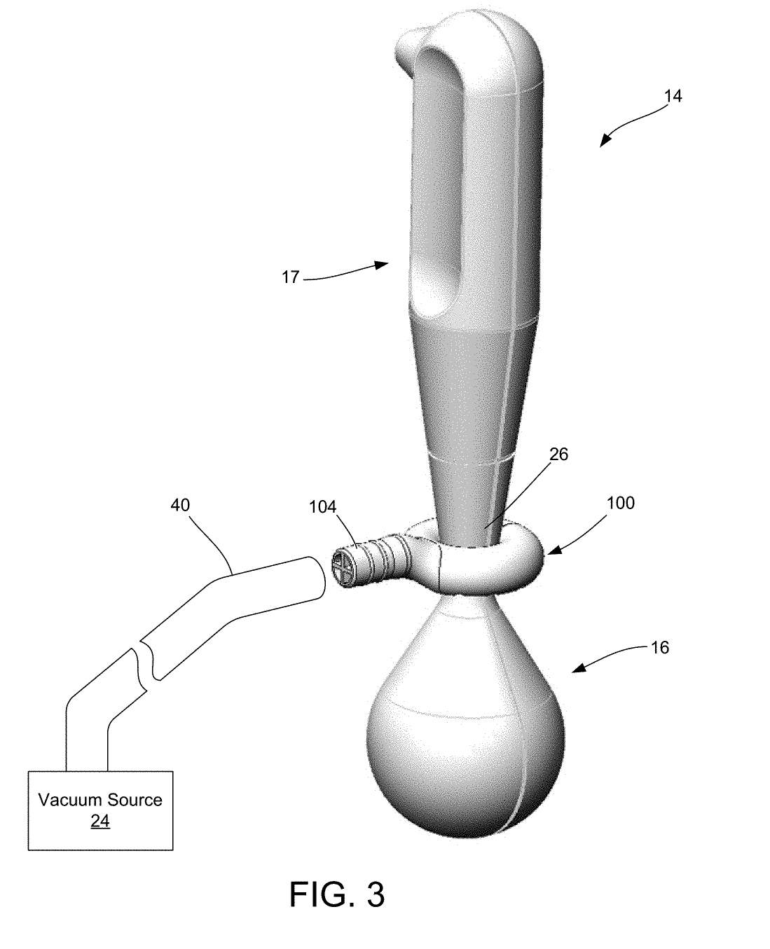

As further shown, the system 10 includes an accessory 100 configured to be mounted to a portion of the ablation device 14, generally adjacent to the distal tip 16 and configured to allow for aspiration of fluids and/or debris during the procedure. The accessory is coupled to a vacuum source 24 over a connection (connection line 40 shown in FIG. 3), as will be described in greater detail herein.

FIG. 2 is a perspective view of the suction collar 100 separated from the exemplary ablation device 14. As shown in the figures and described herein, the ablation device 14 may generally resemble, and function similarly as, the ablation devices described in U.S. application Ser. No. 15/142,616, filed Apr. 29, 2016 (U.S. Publication No. 2016-0317221) and U.S. application Ser. No. 15/337,334, filed Oct. 28, 2016 (U.S. Publication No. 2017/0119454), the entireties of which are incorporated by reference.

Accordingly, the ablation device 14 includes a distal tip 16 and an elongated catheter shaft 17 to which the distal tip 16 is coupled. The catheter shaft 17 is generally configured as a handle and adapted for manual manipulation. The distal tip has a neck portion 26 and a generally spheroid body extending distally from the neck 26. It should be noted that, in some embodiments, the spheroid body may be configured to transition between a collapsed state and an expanded state. For example, the spheroid body may be collapsible to a delivery configuration having a reduced size (e.g., equatorial diameter) relative to the deployed configuration size (e.g., equatorial diameter) of the spheroid body. In some examples, the spheroid body is a generally prolate-spheroid during delivery and transitions to a spheroid shape during deployment. In other embodiments, the spheroid body may be rigid, and thus may maintain a default shape.

The distal tip 16 of the ablation device 14 further includes an electrode array positioned thereon. The electrode array includes at least one conductive member 28. In some embodiments, the electrode array may include a plurality of conductive members 28. The plurality of conductive members 28 may generally transmit RF energy from the ablation generator 20 and can be formed of any suitable conductive material (e.g., a metal such as stainless steel, nitinol, or aluminum). In some examples, the conductive members 28 are metal wires. Accordingly, for ease of description, the conductive member(s) will be referred to hereinafter as “conductive wire(s) 28”.

As illustrated, one or more of the conductive wires 28 can be electrically isolated from one or more of the remaining conductive wires 28. This electrical isolation enables various operation modes for the ablation device 14. For example, ablation energy may be supplied to one or more conductive wires 28 in a bipolar mode, a unipolar mode, or a combination bipolar and unipolar mode. In the unipolar mode, ablation energy is delivered between one or more conductive wires 28 on the ablation device 14 and the return electrode 15, as described with reference to FIGS. 1A and 1B. In bipolar mode, energy is delivered between at least two of the conductive wires 28, while at least one conductive wire 28 remains neutral. In other words, at least, one conductive wire functions as a grounded conductive wire (e.g., electrode) by not delivering energy over at least one conductive wire 28.

As shown, the distal tip 16 may be coupled to the ablation generator 20 and/or irrigation pump 22 at a proximal end 30 of the shaft 17 via an electrical line 32 and a fluid line 36, respectively. Each of the electrical line 32 and fluid line 36 may include an adaptor end 34, 38 configured to couple the associated lines with a respective interface on the ablation generator 20 and irrigation pump 22.

It should be noted that, in some embodiments, the ablation device 14 is configurd to provide RF ablation via a virtual electrode arrangement, which includes distribution of a fluid (from the fluid line 36) along an exterior surface of the distal tip 16 and, upon activation of the electrode array, the fluid may carry, or otherwise promote, energy emitted from the electrode array to the surrounding tissue. For example, the nonconductive distal portion 16 of the ablation device 14 may include an interior chamber configured to receive and retain a fluid (e.g., saline) therein from a fluid source. The distal tip 16 may generally include a plurality of ports or apertures configured to allow the fluid to pass therethrough, or weep, from the interior chamber to an external surface of the distal tip 16. Accordingly, upon positioning the distal tip 16 within a target site (e.g., tissue cavity to be ablated), the electrode array (one or more individual conductive wires 28) can be activated, via user input to the controller 18. The fluid weeping through the perforations (which can be controlled via user input to the controller 18) to the outer surface of the distal tip 16 is able to carry energy from electrode array, thereby creating a virtual electrode. Accordingly, upon the fluid weeping through the perforations, a pool or thin film of fluid is formed on the exterior surface of the distal tip 16 and is configured to ablate surrounding tissue via the RF energy carried from the electrode array.

The accessory 100 is generally in the form of a collar configured to be mounted on the ablation device 14, as shown in FIG. 3, such that, upon activation of a vacuum source 24 coupled thereto, the collar is configured to provide suction at the surgical site (i.e., at the tissue cavity). As will be described in greater detail herein, the accessory (hereinafter referred to as “suction collar 100”) is configured to provide suction for the removal of excess accumulation of steam or smoke, fluid, and/or debris from the surgical site during the treatment procedure and may further be configured to apply a sufficient suction force to a patient's skin surrounding the surgical site to aid in stabilizing the ablation device 14 in place and maintain the distal tip 16 within the tissue cavity during the procedure. As shown in FIGS. 2 and 3, the suction collar 100 includes a ring-shaped body formed from two body members 102a, 102b and configured to be positioned around the shaft 17 of the ablation device 14, and, more specifically around the neck portion 26 adjacent to the distal tip 16. In particular, the ring-shaped body includes at least a first body member 102a forming a first semi-circular half of the body and a second body member 102b forming a second semi-circular half of the body complementary to the first semi-circular half formed by the first body member 102a. Each of the first and second body members 102a, 102b extends from a proximal end 104 of the suction collar 100. The proximal end 104 generally defines a port outlet configured to be coupled to the vacuum source 24 via a connection line 40 (i.e., medical grade tubing or the like). The vacuum source 24 may include a standalone vacuum pump, or, in some embodiments, the vacuum source 24 may include the standard operating room vacuum port to which the connection line 40 can be coupled.

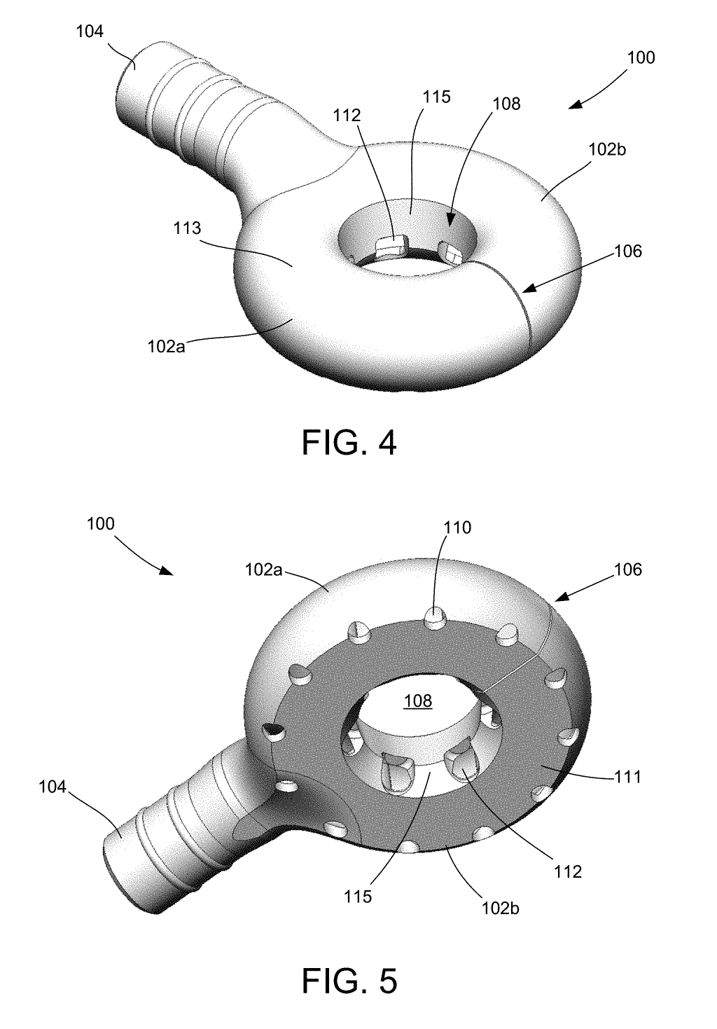

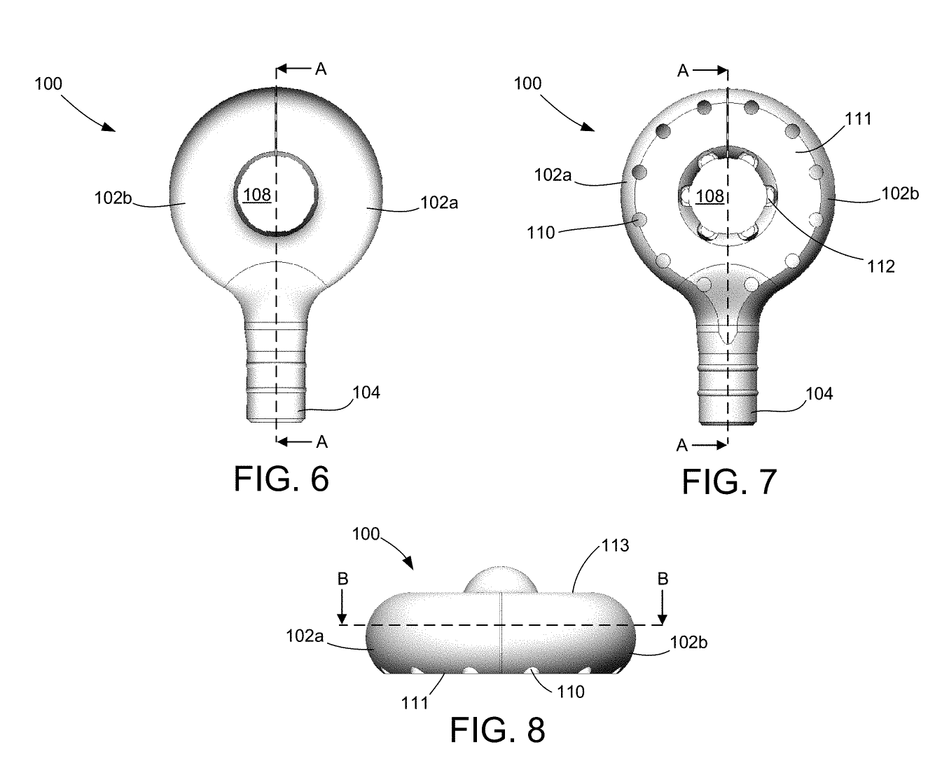

FIGS. 4 and 5 are top and bottom perspective views of the suction collar 100, respectively and FIGS. 6 and 7 are top and bottom plan views of the suction collar 100. FIG. 8 further illustrates a front view of the suction collar 100. As shown, the first body member 102a forms a first semi-circular half of the body and the second body member 102b forms a second semi-circular half of the body complementary to the first semi-circular half. Together, the first and second body members 102a, 102b form an annular body and enclose a central aperture or orifice 108. The central orifice 108 is shaped and/or sized to receive at least a portion of the shaft 17 of the ablation device 14, specifically the neck portion 26 such that the suction collar 100 can be positioned just above the distal tip 16 of the device 14, as shown in FIG. 3.

As shown, the first and second body members 102a and 102b are separated from one another at their respective distal most ends, as indicated by arrow 106 in FIGS. 4 and 5. While the first and second body members 102a, 102b generally extend from and share a common point extending from the proximal end 104, each of the first and second body members 102a, 102b are independent of one another at the respective distal ends (indicated at arrow 106). In other words, the first body member 102a and second body member 102b are not fixed to one another at their respective distal ends and can move relative to one another, which allows for the of suction collar 100 to be releasably coupled to the ablation device 14. For example, the suction collar 100 may be formed form a flexible or deformable material, such as an elastomer, which allows for manipulation and movement of the first and second body members 102a, 102b relative to one another when a user (i.e., surgeon or other medical professional) wishes to attach or remove the suction collar to or from the ablation device 14. In particular, the first and second body members 102a, 102b can separate from one another at least at their respective distal ends upon application of a force thereto (i.e., pressing of the suction collar 100 against the shaft 17 of the device 14 at the separation point 106). In turn, the shaft 17 of the device 14 can then be positioned within the central orifice 108. Once the suction collar 100 is fitted over the shaft 17 (i.e., once the shaft is fully received within the central orifice 108), the first and second body members 102a, 102b will return their default shape (i.e., semi-circular halves) as a result of the shape memory properties of the elastomer material, and the suction collar 100 is securely mounted onto the device 14. The suction collar 100 can be adjusted once mounted onto the shaft 17 of the device 14, which may include rotation of the suction collar 100 about the shaft 17 and/or vertical movement of the suction collar 100 along a length of the shaft 17.

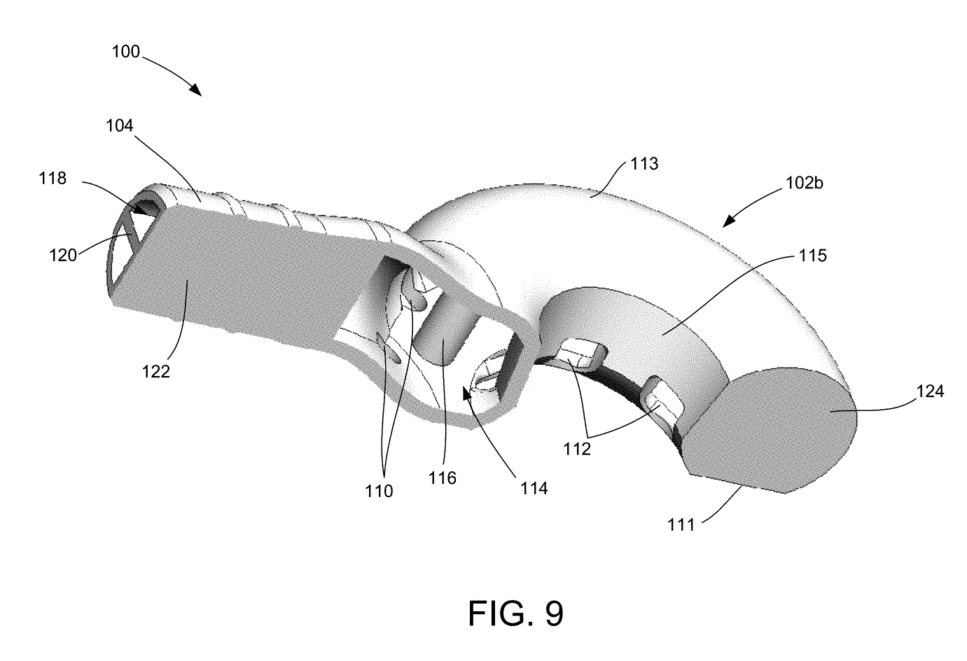

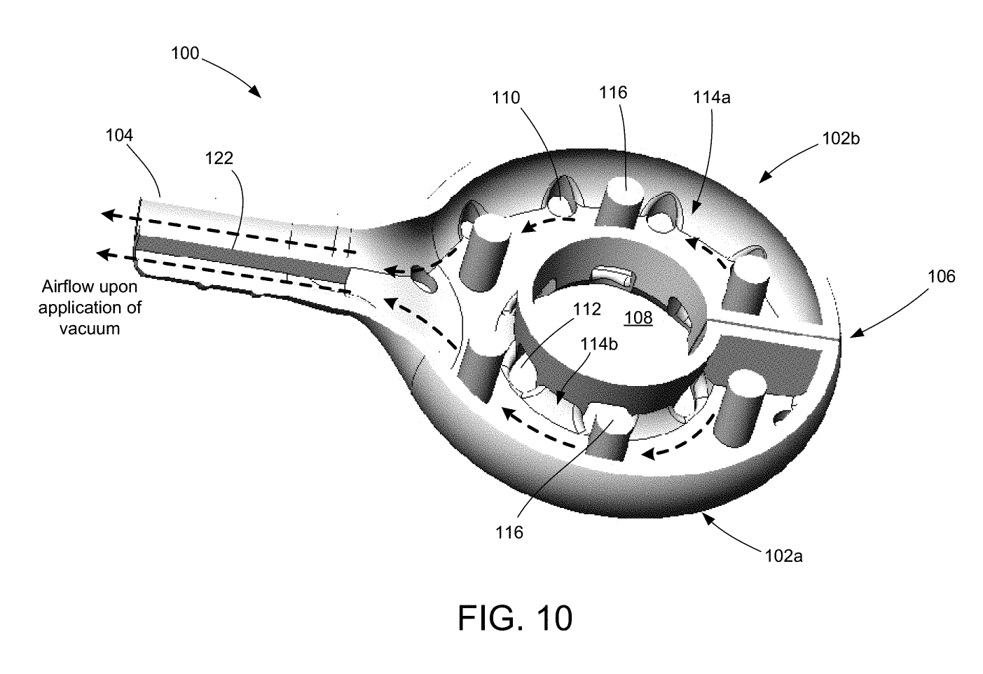

Upon mounting of the suction collar 100 to the ablation device 14, a user need only activate the vacuum source 24, at which point, the suction collar 100 can provide suction to be applied at the surgical site. In particular, the suction collar 100, specifically the first and second body members 102a, 102b, include internal pathways in fluid communication with the port outlet at the proximal end 104 of the suction collar 100. For example, each of the first and second body members 102a, 102b are generally hollow and each includes a pathway extending from a distal end thereof to the port outlet of the proximal end 104 of the suction collar 100 (as shown in FIGS. 9 and 10). The suction collar 100 includes at least a first set of perforations, apertures, or holes 110 defined on a bottom surface 111 thereof. Generally, the bottom surface 111 is configured to be oriented in a direction facing the surgical site, and, in some instances, further engage portions of the surgical site, while a top surface 113 of the suction collar 100 is configured to be oriented in a direction facing away from the surgical site. As shown, the first set of perforations 110 are defined along a circumference of the ring-shaped body, generally arranged in an annular pattern. In some embodiments, the perforations 110 may be positioned equidistantly apart from one another. The suction collar 100 may further include a second set of perforations 112 generally defined on an inner circumference 115 of the first and second body members 102a, 102b and adjacent to the bottom surface 111.

Each of the perforations 110 and 112 are in fluid communication with the internal pathways of the first and second body members 102a, 102b, and, in turn, in fluid communication with the port outlet at the proximal end 104 of the suction collar 100. For example, FIG. 9 is a perspective sectional view of the suction collar 100 of FIGS. 6 and 7 taken along lines A-A illustrating the internal pathways from the perforations 110, 112 to the port outlet. FIG. 10 is a perspective section view of the suction collar of FIG. 8 taken along lines B-B illustrating the internal pathways from the perforations 110, 112 to the port outlet.

As shown, each of the first and second body members 102a, 102b includes an internal pathway 114a, 114b, respectively. Each of the first and second body members 102a, 102b further includes one or more support structures or pillars 116 positioned within the internal hollow bodies and extending from an internal bottom surface to an internal top surface of the first and second body members 102a, 102b. The support pillars 116 are configured to maintain the hollow nature of each and maintain the open internal pathways 114a, 114b and prevent collapsing of a body member upon itself due to the vacuum. The port outlet at the proximal end 104 generally includes an outlet pathway 118 in fluid communication with the internal pathways 114a, 114b. As shown, in some embodiments, one or more support structures may also be provided in the port outlet, specifically within the outlet pathway 118 to maintain the integrity of the pathway 118 and prevent collapsing of the pathway 118 due to the vacuum applied thereto. For example, at least a first support structure 120 may extend across an inner diameter of the proximal end 104 and lie along a plane that is substantially parallel to a plane upon which the ring-shaped body lies. The suction collar 100 may further include a second support structure 122 that extends across the inner diameter of the proximal end and traverses the first support structure 120, generally lying along a plane that is substantially perpendicular to the plane upon which the ring-shaped body lies. As shown, each of the first and second body members 102a, 102b includes a distal end 124 that generally meet one another when the suction collar 100, specifically the first and second body members 102a, 102b are in a default shape.

Accordingly, upon activation of the vacuum source 24, suction is created at each of the perforations 110, 112, and, in turn, fluid, steam, smoke, and/or debris from the surgical site may then flow into the suction collar 100, by way of one or more of the perforations 110, 112, flow through the pathways 114a, 114b of at least one of the first and second body members 102a, 102b, and eventually flow into the outlet pathway 118 and out of the suction collar 100 via the port outlet at the proximal end 104, as indicated by the arrows.

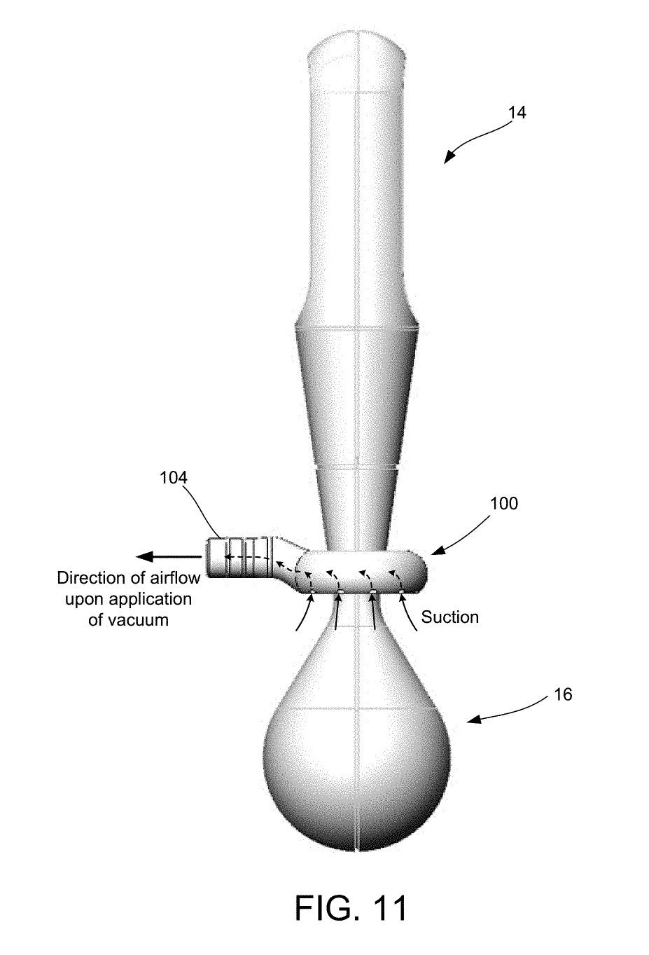

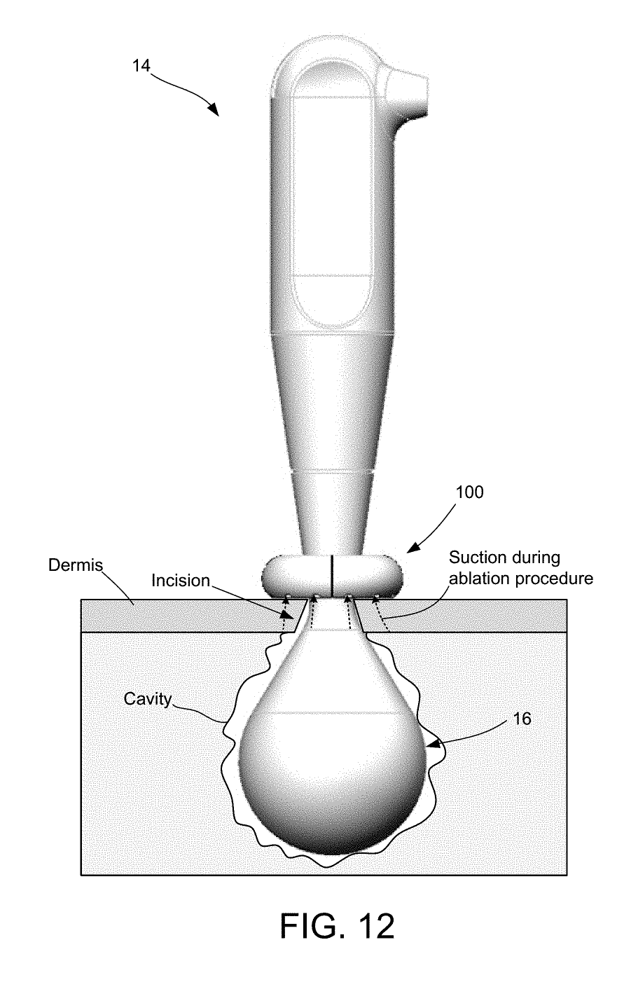

FIG. 11 is a side view of the suction collar 100 mounted to the ablation device 14 and illustrating suction via the perforations of the suction collar 100. FIG. 12 illustrates placement of the distal tip 16 of the ablation device 14 within a tissue cavity and positioning of the suction collar 100 adjacent to the surgical site for subsequent aspiration of fluids, steam, smoke, and/or debris from the surgical site and/or stabilizing of the ablation device 14 based on suction of the skin surrounding the incision of the tissue cavity. For example, in addition to aspirating fluid, steam, smoke, and/or debris, the suction collar 100 may further be configured to apply a sufficient suction force to a patient's skin surrounding the surgical site to aid in stabilizing the device 14 in place and maintain the distal tip 16 of the device 14 within the surgical site during the procedure. For example, in the event that a surgeon is ablating marginal tissue within a cavity with an ablation device 14, as shown in FIG. 12, the surgeon can utilize the suction collar 100 to not only minimize steam, smoke, saline, as well as debris, such a liquefied fat, from the ablation site (i.e., tissue cavity), but to further stabilize and maintain the distal tip 16 within the cavity as a result of suction applied to the patient's skin surrounding the incision leading to the cavity. For example, the bottom surface 111 of the body of the suction collar 100 can directly engage the patient's skin, and, upon activation of the vacuum source 24, suction at the first set of perforations 110 may be sufficient to hold the suction collar 100 in place, and, in turn, stabilizes the ablation device 100.

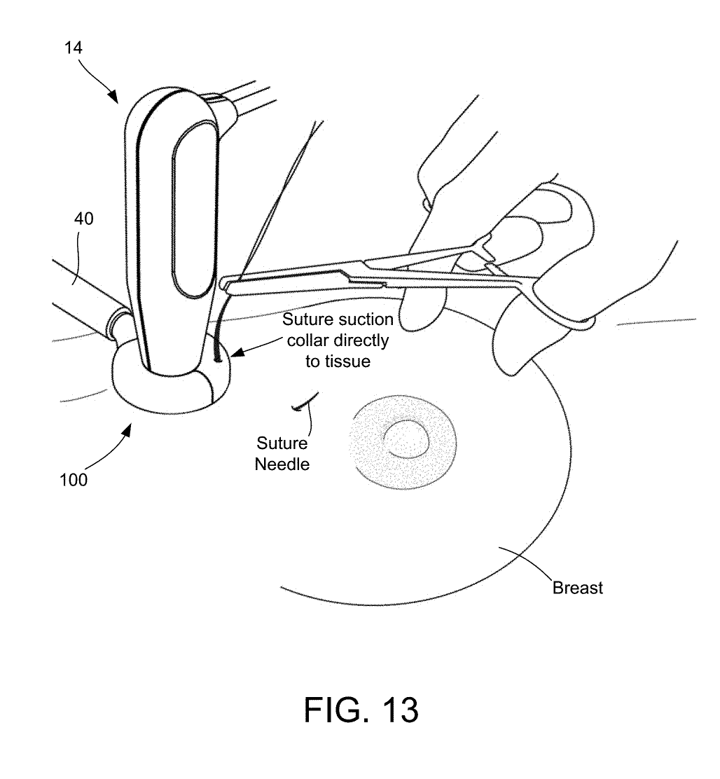

FIG. 13 illustrates placement of the distal tip 16 within a tissue cavity and positioning of the suction collar 100 adjacent to the surgical site and further illustrates suturing of the suction collar 100 to the surrounding skin. As previously described, the suction collar 100 is made from a flexible or deformable material having a relatively low durometer value. Accordingly, in some embodiments, a surgeon or other medical professional may suture the suction collar 100 directly to the patient's skin during a procedure, in that the softness of the suction collar material allows for a needle to pierce the suction collar and suturing to occur, so as to further aid holding the ablation device 14 in place.

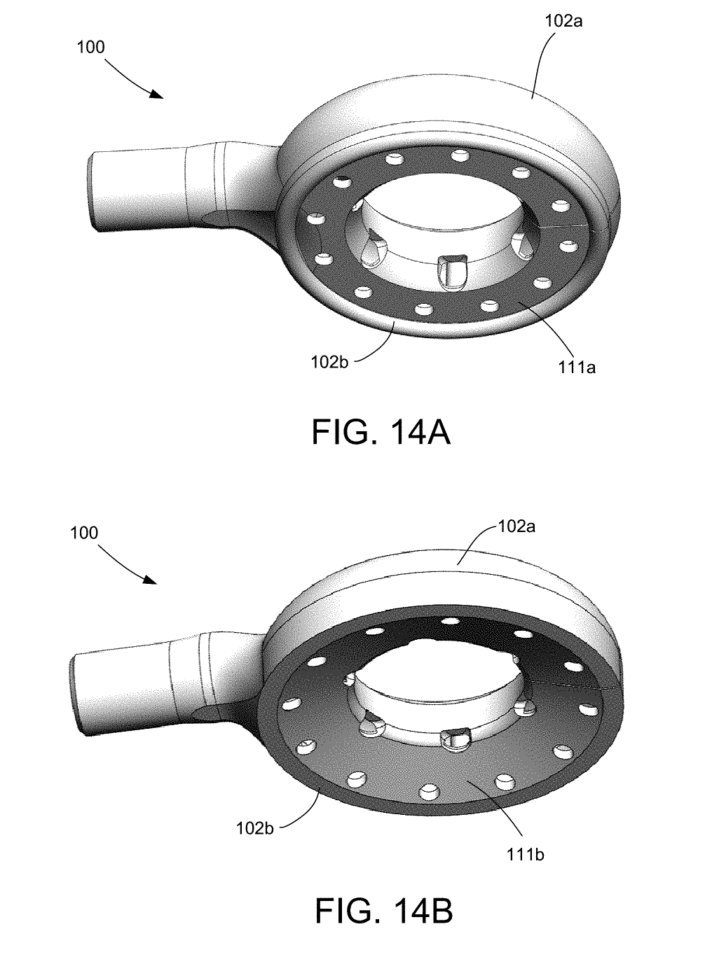

It should be noted that, in some embodiments, the suction collar 100 may have different configurations depending on the particular application and body part upon which the ablation procedure (or any other surgical procedure) is to be performed. For example, FIGS. 14A and 14B are bottom perspective views of a suction collar 100 having different bottom surface contours. For example, as shown in FIG. 14A, the suction collar 100 includes a substantially planar or flat bottom surface 111a, which may be useful when procedures are performed in areas in which the surgical site is relatively flat. However, as shown in FIG. 14B, the suction collar 100 includes a substantially concave bottom surface 111b, which may be particularly useful when the surgical site has a rounded surface, such as the case with breast tissue or the like. The different bottom surface contours allows for improved suction on corresponding surfaces.

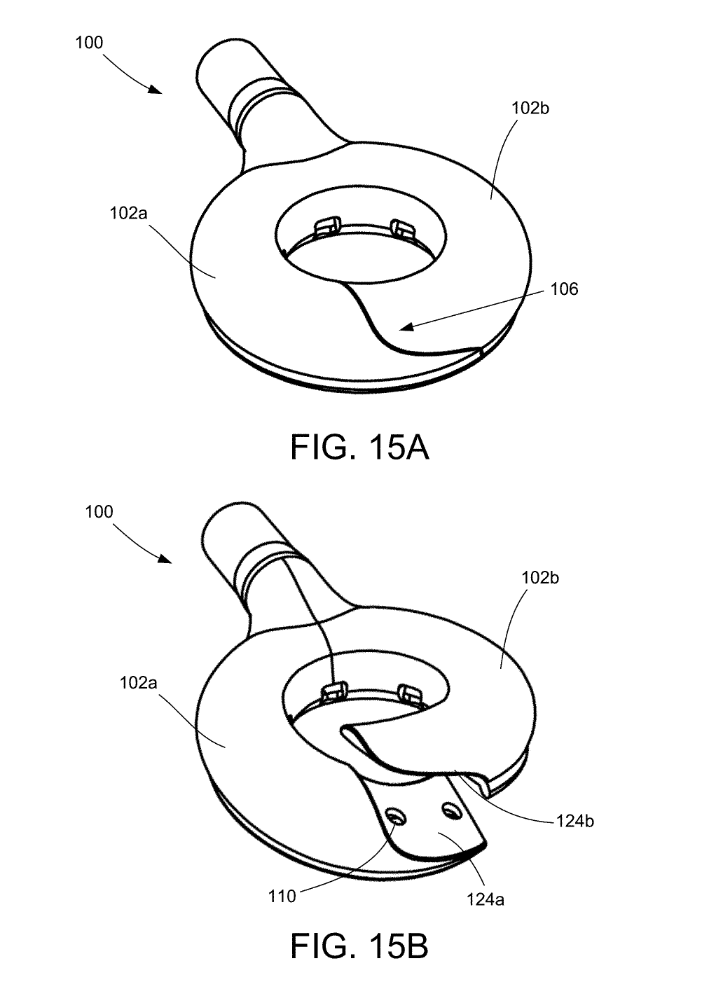

As previously described, the first body member 102a and second body member 102b of the suction collar 100 are not fixed to one another at their respective distal ends 124a and 124b, respectively, and thus can move relative to one another, as shown in FIGS. 15A and 15B. In particular, FIGS. 15A and 15B are top perspective views of a suction collar 100 illustrating one embodiment of the cooperating engagement between distal ends 124a, 124b of the first and second body members 102a, 102b and the separation of each from one another. Due to the independent construction of the first and second body members 102a, 102b, the suction collar 100 is able to be releasably coupled to the ablation device 14. In particular, the first and second body members 102a, 102b can separate from one another at least at their respective distal ends 124a, 124b upon application of a force thereto (i.e., pressing of the suction collar 100 against the shaft 17 of the device 14 at the separation point 106). As shown, unlike the substantially linear separation point 106 of the collar 100 shown in FIGS. 4 and 5, for example, the separation point of the collar 100 illustrated in FIGS. 15A and 15B has an arcuate or curved design, in that the distal ends 124a, 124b of the first and second body members 102a, 102b engage one another in an overlapping manner. In particular, the distal end 124b of the second body member 102b is shown overlapping the distal end 124a of the first body member 102a, such that, in a default position, the distal ends 124a, 124b engage one another, such that corresponding perforations 110 defined at the distal ends 124a, 124b from the first and second body members 102a, 102b are aligned with one another and thus function as a single unit. However, the distal ends 124a, 124b can be separated for subsequent mounting or removal of the suction collar to or from the ablation device 14.

For example, when mounting the suction collar 100 to the device 14, a user need only press the suction collar against the shaft 17 of the device 14, such that initial contact is made between the suction collar 100 and the shaft 17 at the separation point where the two distal ends 124a, 124b of the first and second body members 102a, 102b meet (indicated at arrow 106). Due to the elastic nature of the elastomer material, at least one of the first and second body members 102a, 102b is able to deform to a degree and thereby separate from the other body member, which, in turn, exposes the central orifice 108 and allows for the shaft 17 of the device 14 to be inserted within. Once the suction collar 100 is fitted over the shaft 17 (i.e., once the shaft is fully received within the central orifice 108), the first and second body members 102a, 102b will return their default shape (i.e., semi-circular halves) as a result of the shape memory properties of the elastomer material, and the suction collar 100 is securely mounted onto the device 14. In turn, the perforations 110 defined on the opposing distal ends 124a, 124b will align with one another and will subsequently function as a single unit for subsequent suction.

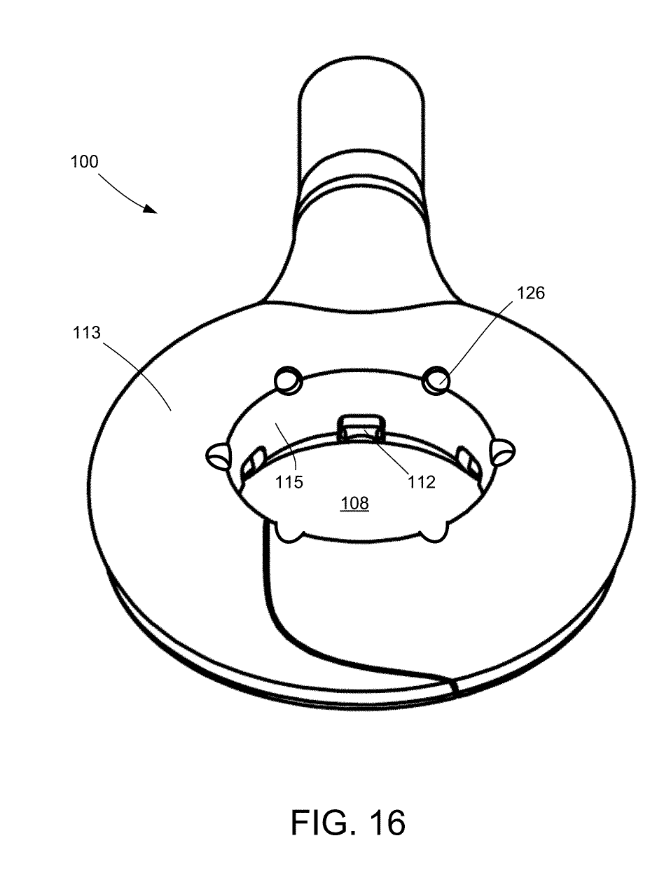

FIG. 16 is a perspective top view of a suction collar 100 having at least a third set of perforations 126 defined along the inner circumference 115 of the first and second body members 102a, 102b and further adjacent to the top surface 113 of the suction collar 100. Similar to the first and second sets of perforations 110, 112, the third set of perforations 126 may be positioned equidistantly apart from one another around the circumference of the ring-shaped body. Similar to perforations 110 and 112, each of the third set of perforations 126 are in fluid communication with the internal pathways of the first and second body members 102a, 102b, and, in turn, in fluid communication with the port outlet at the proximal end 104 of the suction collar 100 and thus may provide suction upon activation of the vacuum source 24.

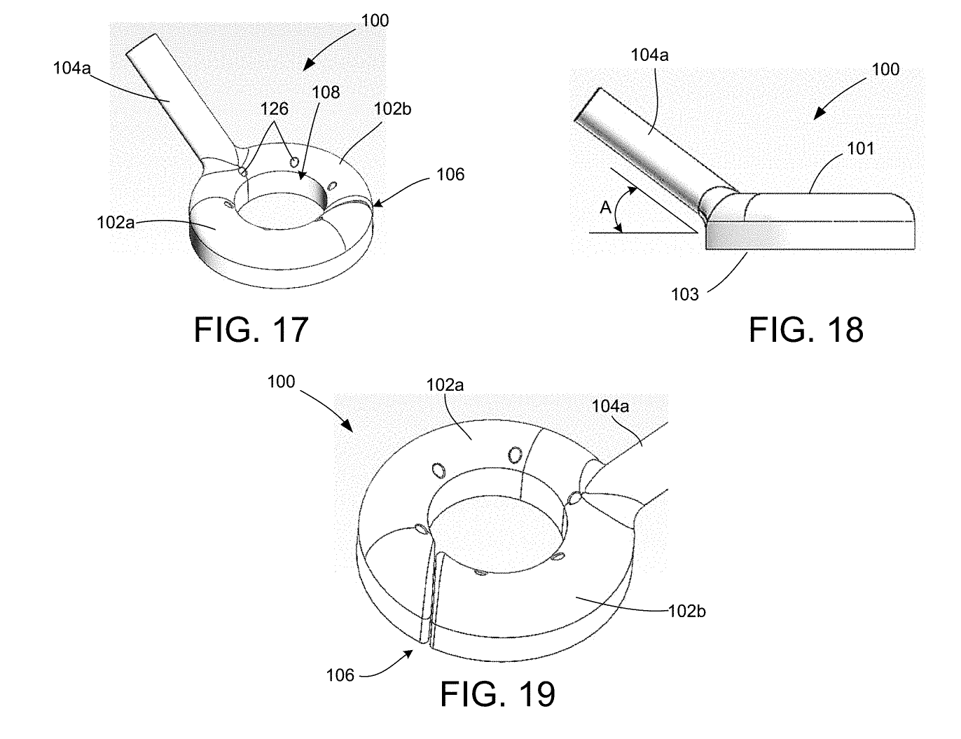

It should be noted that, in some embodiments, the suction collar 100, specifically the body member of the suction collar 100, may include a two-piece molded construction, as shown in FIGS. 17-21, as opposed to a single, molded construction, as illustrated in FIGS. 1-16. Furthermore, as previously described, the first body member 102a and second body member 102b of the suction collar 100 are not fixed to one another at their respective distal ends 124a and 124b, respectively, and may include a non-radial, substantially linear separation there between, as generally illustrated in FIGS. 17-21, as opposed to an overlapping separation, previously described herein.

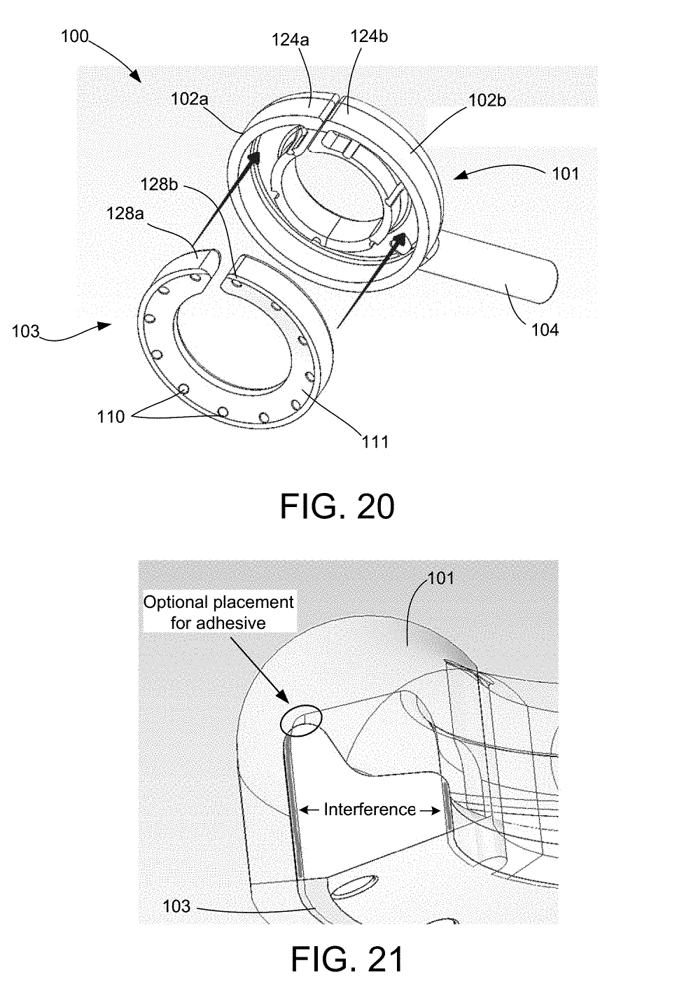

For example, FIGS. 17 and 18 are top perspective and side views, respectively, of a suction collar 100 having a body member comprised of a two-piece molded construction. FIG. 19 is an enlarged top perspective view of the suction collar 100 illustrating a non-radial, substantially linear separation 106 between distal most ends 124a, 124b of first and second body members 102a, 102b. FIG. 20 is a bottom exploded perspective view of the suction collar 100 illustrating the two-piece molded construction, specifically an upper member 101 and a lower member 103 separated from one another. The two-piece, molded construction of the suction collar may improve the ease of manufacturing of the suction collar, as opposed to a single, one-piece construction.

As shown, the first body member 102a forms a first semi-circular half of the body and the second body member 102b forms a second semi-circular half of the body complementary to the first semi-circular half. Together, the first and second body members 102a, 102b form an annular body and enclose a central aperture or orifice 108. The central orifice 108 is shaped and/or sized to receive at least a portion of the shaft 17 of the ablation device 14, specifically the neck portion 26 such that the suction collar 100 can be positioned just above the distal tip 16 of the device 14, as previously shown in FIG. 3.

The first and second body members 102a and 102b are separated from one another at their respective distal most ends, as indicated by arrow 106. While the first and second body members 102a, 102b generally extend from and share a common point extending from the proximal end 104, each of the first and second body members 102a, 102b are independent of one another at the respective distal ends (indicated at arrow 106). In other words, the first body member 102a and second body member 102b are not fixed to one another at their respective distal ends and can move relative to one another, which allows for the of suction collar 100 to be releasably coupled to the ablation device 14. For example, the suction collar 100 may be formed form a flexible or deformable material, such as an elastomer, which allows for manipulation and movement of the first and second body members 102a, 102b relative to one another when a user (i.e., surgeon or other medical professional) wishes to attach or remove the suction collar to or from the ablation device 14. In particular, the first and second body members 102a, 102b can separate from one another at least at their respective distal ends upon application of a force thereto (i.e., pressing of the suction collar 100 against the shaft 17 of the device 14 at the separation point 106).

As previously described, the suction collar 100, specifically the first and second body members 102a, 102b, include internal pathways in fluid communication with the port outlet at the proximal end 104a of the suction collar 100. In the illustrated embodiment of FIG. 18, the proximal end 104a generally extends from the body member at an angle A in an offset manner, thereby allowing for the body member of the suction collar 100 to engage a skin surface while the proximal end generally rests a sufficient distance from the skin surface to improve the ease with which a procedure can be performed.

FIG. 20 is a bottom exploded perspective view of the suction collar 100 illustrating the two-piece molded construction of the body. FIG. 21 is a perspective view, partly in section, illustrating the coupling of the upper and lower members 101, 103 to one another to form the body of the suction collar 100.

As shown, the body generally includes an upper member 101, generally forming a top half of the body of the suction collar, and a lower member 103, generally forming a lower half of the body of the suction collar. At least a portion of the lower member 103 is configured to be received within a portion of the upper member 101 and reside therein. When coupled to one another, the upper and lower members 101, 103 form an assembled suction collar assembly. For example, each of the first and second body members 102a, 102b are generally hollow and each includes a pathway extending from a distal end 124a, 124b thereof to the port outlet of the proximal end 104a of the suction collar 100. The lower member 103 is generally annular in shape and corresponds to the shape of the first and second body members 102a, 102b. For example, the lower member 103 may generally include an annular shape with two distal ends 128a, 128b, generally corresponding to the distal most ends of the first and second body members 102a, 102b, thereby allowing for the lower member 103 to correspondingly fit with the first and second body members 102a, 102b of the upper member 101. The lower member 103 generally includes at least a first set of perforations, apertures, or holes 110 defined on a bottom surface 111 thereof. Generally, the bottom surface 111 is configured to be oriented in a direction facing the surgical site, and, in some instances, further engage portions of the surgical site, while the upper member 101, specifically a top surface 113 thereof, is configured to be oriented in a direction facing away from the surgical site. As shown, the first set of perforations 110 are defined along a circumference of the ring-shaped body of the lower member 103, generally arranged in an annular pattern. In some embodiments, the perforations 110 may be positioned equidistantly apart from one another. The suction collar 100 may further include a second set of perforations 126 generally defined on the top surface 113 of the upper member 101. The second set of perforations 126 may be positioned equidistantly apart from one another around the circumference of the ring-shaped body. Each of the first and second sets of perforations 110, 126 are in fluid communication with the internal pathways of the first and second body members 102a, 102b, and, in turn, in fluid communication with the port outlet at the proximal end 104a of the suction collar 100 and thus may provide suction upon activation of the vacuum source 24.

The upper and lower members 101, 103 may be coupled to one another via an interference fit. However, it should be noted that the upper and lower members 101, 103 may be coupled to one another by any other known methods, including, but not limited to, an adhesive (e.g., an epoxy of the like) and welding (e.g., ultrasonic welding). The upper and lower members 101, 103 may be constructed of the same material. However, in some embodiments, the upper and lower members 101, 103 may be constructed of different materials. For example, in one embodiment, the lower member 103 may be constructed of a material having a durometer that is greater than a durometer value of a material of the upper member 101. In other words, the lower member 103 may be constructed of a stiffer, more rigid material while the upper member 101 may be constructed of a softer, less rigid material. Accordingly, when coupled to one another, the upper member 101, due to being constructed from a softer material, may be able to better conform to the shape of the lower member 103 and improve the fit between the two. In some embodiments, an annular ring of epoxy or other type of adhesive may be applied between the upper and lower members 101, 103 to further fix the components to one another, as illustrated in FIG. 21.

The upper and lower members 101, 103 may be constructed of a medical grad elastomer, including, but not limited to, ethylene-propylene (EP, EPT, EPDM), fluorcarbon (Viton, FKM), and silicone (VMQ), for example.

It should be noted that, in some embodiments, it is contemplated that a single suction collar will be packaged and delivered to the user. However, in some embodiments, it is contemplated that a vacuum kit may be provided, which may include the suction collar, the tubing set, configured to be coupled to the suction collar, via an adapter or coupling member, and a vacuum source, such as a standard vacuum source found in an operating room in a hospital or other facility.

The suction collar of the present disclosure provides numerous advantages. The suction collar facilitates the ease of operation and makes it possible for a single user to perform certain tasks or treatments without the help of an assistant. In particular, some known medical procedures may often require an assistant (i.e., fellow surgeon, physician assistant, nurse, etc.) to handle and manipulate additional instruments during a procedure, while the surgeon handles electrosurgical device which may require the surgeon to use both hands for the treatment. The suction collar is an accessory configured to be releasably mounted on the electrosurgical device, specifically mounted adjacent to the working end (i.e., applicator head) of the device, thereby allowing for aspiration of fluid, steam, smoke, and/or debris in an efficient manner, as the suction collar movement directly corresponds to movement and manipulation of the electrosurgical device. The suction collar further provides for stabilization of the electrosurgical device, thereby providing improved accuracy and safety during an ablation procedure. Furthermore, because the suction collar is not integrally formed as part of the electrosurgical device itself, a surgeon can simply remove the suction collar when aspiration and/or stabilization of the electrosurgical device is not desired. Accordingly, a suction collar consistent with the present disclosure may be well suited for use with electrosurgical devices, particularly for use with ablation devices that may rely on the use of conductive fluids (e.g., saline) for the ablation procedure and that will further generate steam, vapors, and smoke from heated or burnt tissue, specifically marginal tissue that has been ablated within a cavity an effort to manage residual disease within.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A suction collar for an electrosurgical device, the suction collar comprising:
a hollow, ring-shaped body configured to be releasably mounted to an electrosurgical device, the hollow, ring-shaped body comprising one or more perforations defined thereon and providing a passageway from an exterior surface of the body to an interior chamber of the body; and
a proximal end extending from the body, the proximal end defining an outlet in fluid communication with the chamber of the body such that a pathway is defined extending between the one or more perforations on the body and the outlet of the proximal end, wherein the outlet is configured to be coupled to a vacuum source to thereby provide suction at the one or more perforations.

2. The suction collar of claim 1, wherein the hollow, ring-shaped body comprises:
a first body member forming a first semi-circular half of the body;
a second body member forming a second semi-circular half of the body complementary to the first semi-circular half of the body; and
a central orifice formed by and substantially enclosed within the inner circumferences of the first and second body members.

3. The suction collar of claim 2, wherein the central orifice is shaped and/or sized to receive a shaft portion of the electrosurgical device.

4. The suction collar of claim 2, wherein each of the first and second body members extend from the proximal end of the suction collar and each further comprises a distal end.

5. The suction collar of claim 4, wherein at least the distal ends of the first and second body members are configured to move independent of one another.

6. The suction collar of claim 5, wherein each of the first and second body members is configured to transition from a default state to a deformed state upon application of a force thereto and return to the default state upon removal of the compression force therefrom.

7. The suction collar of claim 6, wherein, when in the default state, the first and second body members cooperatively form a substantially annular body and the distal ends are in alignment with one another, and, when in a deformed state, a distal end of at least one of the body members is misaligned relative to the corresponding distal end of the other body member.

8. The suction collar of claim 5, wherein the distal ends of the first and second body members are configured to separate from one another to thereby expose the central orifice for insertion of a shaft portion of an electrosurgical device.

9. The suction collar of claim 5, wherein the first and second body members comprise a flexible material.

10. The suction collar of claim 9, wherein the first and second body members comprise and elastomeric material or shape memory material.

11. The suction collar of claim 2, wherein the first and second body members comprise a first set of perforations defined on a bottom surface thereof.

12. The suction collar of claim 11, wherein:
each of the first set of perforations defined on the first body member provides a passageway from an exterior surface of the first body member to an interior chamber of the first body member; and
each of the first set of perforations defined on the second body member provides a passageway from an exterior surface of the second body member to an interior chamber of the second body member.

13. The suction collar of claim 12, wherein the proximal end outlet is in fluid communication with the interior chambers of the first and second body members such that pathways are defined extending between the first set of perforations the proximal end outlet.

14. The suction collar of claim 7, wherein the first and second body members further comprise a second set of perforations defined on inner circumferences of each of the first and second body members.

15. The suction collar of claim 14, wherein:
each of the second set of perforations defined on the first body member provides a passageway from an exterior surface of the first body member to an interior chamber of the first body member; and
each of the second set of perforations defined on the second body member provides a passageway from an exterior surface of the second body member to an interior chamber of the second body member.

16. The suction collar of claim 15, wherein the proximal end outlet is in fluid communication with the interior chambers of the first and second body members such that pathways are defined extending between the second set of perforations and the proximal end outlet.

17. The suction collar of claim 14, wherein, upon activation of the vacuum source, at least one of the perforations of the first and second sets of perforations is configured to provide suction at a surgical site for aspiration of at least one of steam, smoke, fluid, and debris during a treatment procedure with the electrosurgical device.

18. The suction collar of claim 14, wherein, upon activation of the vacuum source, at least one of the perforations of the first and second sets of perforations is configured to provide suction at a surgical site for applying the vacuum to a patient's skin to thereby stabilize the electrosurgical device in place.

19. The suction collar of claim 1, wherein, upon activation of the vacuum source, the one or more perforations defined on the hollow, ring-shaped body, is configured to provide suction at a surgical site at least one of aspiration of steam, smoke, fluid, or debris during a treatment procedure with the electrosurgical device and application of the vacuum to a patient's skin to thereby stabilize the electrosurgical device in place.

20. The suction collar of claim 1, wherein the hollow, ring-shaped body comprises a sufficiently lower durometer material configured to be pierced via a suturing needle and subsequently be sutured to a surgical site on a patient.
